(19) United States
(12) Patent Application Publication
(10) Pub. No.: US 2026/0283463 A1
(43) Pub. Date: Sep. 24, 2026

(54) SYSTEMS AND METHODS FOR RETINAL IMAGING

(71) Applicants: Wei-Chiang Lin, Miami, FL (US); Shuliang Jiao, Miami, FL (US); Noble Amadi, Miami, FL (US); Rui Zhou, Miami, FL (US)

(72) Inventors: Wei-Chiang Lin, Miami, FL (US); Shuliang Jiao, Miami, FL (US); Noble Amadi, Miami, FL (US); Rui Zhou, Miami, FL (US)

(73) Assignee: The Florida International University Board of Trustees, Miami, FL (US)

(21) Appl. No.: 19/573,521

(22) Filed: Mar. 20, 2026

Related U.S. Application Data
(60) Provisional application No. 63/775,589, filed on Mar. 21, 2025.

(51) Int. Cl. A61B 3/14; A61B 3/00; A61B 3/12

(52) U.S. Cl. CPC A61B 3/14; A61B 3/0025; A61B 3/0091; A61B 3/12

(57) ABSTRACT

Systems and methods for retinal imaging are provided for identifying biomarkers of eye and brain diseases. The system enables imaging of retinal and choroidal vasculature, transpalpebral near-infrared (NIR) illumination for expanded retinal coverage, and high-speed spectral imaging to extract temporal blood oxygenation data. Local optical stimulation can assess functional characteristics of the retina to identify early-stage biomarkers. The system includes a programmable illuminator comprising a visible and near-infrared (VIS-NIR) light-emitting diode (LED) array and a liquid crystal display (LCD) panel for adjustable fixation targets and point-illumination patterns. The system can improve retinal imaging devices and ophthalmic diagnostic instruments, by enabling precise alignment, high-resolution imaging, functional vascular assessment, and enhanced early detection of ocular pathologies.

### CROSS-REFERENCE TO RELATED APPLICATION

This application claims the benefit of U.S. Provisional Application Ser. No. 63/775,589, filed Mar. 21, 2025, the disclosure of which is hereby incorporated by reference in its entirety, including all figures, tables, and drawings.

### BACKGROUND

Retinal imaging provides a critical, non-invasive means for evaluating ocular and systemic health, enabling early detection of conditions like diabetic retinopathy, glaucoma, macular degeneration, diabetes, hypertension, and cardiovascular disorders. Owing to the retina's shared embryological origin with the brain and its closely analogous microvascular architecture, retinal imaging further offers an opportunity for early detection of neurological disorders including Alzheimer's disease, Parkinson's disease, and multiple sclerosis. However, despite this broad diagnostic potential, existing retinal imaging technologies remain constrained in their ability to reliably visualize both retinal and choroidal ocular structures with sufficient contrast, resolution, and patient tolerance. Because these benchtop systems require a stationary, clinic-mounted setup with tightly controlled lighting and stable patient positioning, their use is largely restricted to specialized ophthalmic facilities and excludes patients who cannot maintain an upright posture. As a result, their use is largely confined to dedicated ophthalmic clinics, reducing opportunities for broader screening, limiting access in community and bedside settings, and making frequent monitoring less feasible for many patients.

Fundus cameras are widely used to capture images of retina for diagnostic and screening purposes. However, existing systems predominantly rely on visible-light illumination, which is optimized for retina but offers limited penetration and contrast for choroid. Prolonged exposure to visible light may also cause patient discomfort or reduced compliance. While near-infrared (NIR) illumination has been introduced in some systems to address these issues, such implementations are often limited to separate imaging modes or optical paths, increasing system complexity, alignment requirements, and cost, while reducing workflow efficiency. Further, many current fundus cameras and other handheld devices generally provide lower image quality and a narrower field of view than benchtop systems. Their performance is more sensitive to operator skill, making them susceptible to misalignment, motion blur, and uneven illumination. In addition, current handheld platforms are limited to anatomical imaging, constraining their utility for functional retinal assessments.

### BRIEF SUMMARY

Embodiments of the subject invention provide novel and advantageous systems, devices, and methods for retinal imaging, designed (and/or configured) to explore new biomarkers for eye and brain diseases. Embodiments can have simultaneous high-resolution imaging of both retinal and choroidal vasculature and/or high-speed spectral imaging that extracts critical temporal data on regional blood oxygenation. Additionally, embodiments can facilitate precise local stimulation to study functional characteristics of the retina, potentially revealing new biomarkers for early disease detection. A system/device of an embodiment of the subject invention can be referred to as an advanced functional fundus camera. The system/device can be configured to acquire images from both eyes sequentially or simultaneously. Also, the system/device can create a controlled dark environment for the patient, thereby facilitating examinations that traditionally require a dedicated darkroom.

In an embodiment, a system for capturing non-mydriatic fundus images of retinal vasculature and choroidal vasculature with adjustable illumination intensity can comprise: a programmable illuminator including an optical assembly. The optical assembly can comprise: a backlight assembly comprising a visible and near-infrared (VIS-NIR) light-emitting diode (LED) array, the VIS-NIR LED array comprising red LED channel, green LED channel, blue LED channel, and near-infrared (NIR) LED channel configured to provide wavelength-selective illumination; diffuser plates or diffuser films configured to homogenize illumination emitted by the VIS-NIR LED array and provide spatially uniform illumination across an illumination aperture; and a liquid crystal display (LCD) panel. The LCD panel can comprise: a first linear polarizer configured to receive the diffused illumination; a thin-film transistor (TFT) layer adjacent to the first linear polarizer and configured to apply spatially resolved electric fields; a liquid crystal layer adjacent to the TFT layer and configured to modulate polarization state and transmissivity of incident light in response to control signals applied via the TFT layer; and a second linear polarizer positioned after the liquid crystal layer and configured to convert polarization modulation induced by the liquid crystal layer into intensity modulation of transmitted light. The LCD panel can be configured to selectively transmit the red LED channel, the green LED channel, and/or the blue LED channel from the VIS-NIR LED array to generate adjustable fixation targets comprising one or more point-illumination patterns. The system can further comprise: an image sensor comprising a camera; a digital input/output (DIO) interface configured to operably couple the programmable illuminator with a controller; and the controller. The controller can comprise: one or more processors; and a non-transitory machine-readable medium in operable communication with the one or more processors and having instructions stored thereon that, when executed by the one or more processors, cause the system to operate the programmable illuminator in a Standby Mode, a Pre-Imaging/Alignment Mode, and an Imaging Mode, the Imaging Mode comprising a VIS Imaging Mode Type and an NIR Imaging Mode Type. The Standby Mode can comprise a deactivated state of all VIS LED channels, NIR LED channel, and illuminator pixels, in which there is a complete absence of optical output from the optical assembly. The Pre-Imaging/Alignment Mode can comprise an activated state of the NIR LED channel and the red LED channel, the red LED channel being configured to pass through activated pixels of the LCD panel, reach a retina of a subject, and be visible to the subject, the NIR LED channel not being blocked by the LCD panel, such that the NIR illumination passes through the LCD panel and illuminate the entire retina. The VIS Imaging Mode Type can comprise: an activated state of the VIS LED channels with the NIR LED channel being deactivated; the activated VIS LED channels being pulsed; illumination from the pulsed VIS LED channels being delivered to a retina for a duration of about 200 milliseconds or less; and the camera, with an NIR filter, recording an image of retinal vasculature. An operator can control duration and power of the pulsed VIS LED channels via the controller to adjust brightness, contrast, and/or overall quality of the image. The NIR Imaging Mode Type can comprise: an activated state of only the NIR LED channel with the VIS LED channels being deactivated; the activated NIR LED channel being pulsed or continuous (i.e., not being pulsed); and the camera, without an NIR filter, recording an image of choroidal vasculature. The liquid crystal layer can be transparent to wavelengths above 750 nanometers (nm) (or about 750 nm), thereby permitting illumination from the NIR LED channel to pass without significant attenuation during the Pre-Imaging/Alignment Mode while the LCD panel generates visible-light fixation targets. The system can further comprise a stimulation subsystem configured to apply localized optical stimulation to retinal tissue or choroidal tissue, the controller being configured to coordinate the localized optical stimulation with image acquisition to assess functional characteristics of the retina, the assessment enabling identification of biomarkers associated with early-stage ocular disease. The camera (e.g., a wearable fundus camera) can also operate using either transpalpebral illumination or trans-pars plana illumination to expand the retinal region that can be imaged. The image sensor and the controller can be configured to perform high-speed spectral imaging, the high-speed spectral imaging can be configured to extract local hemodynamic information from retinal vasculature or choroidal vasculature, and the local hemodynamic information can comprise regional blood oxygenation as a function of time. The controller can be configured to store image data acquired across multiple imaging sessions, and the stored image data can be used to detect temporal changes in retinal vasculature or choroidal vasculature.

In another embodiment, a method for capturing non-mydriatic fundus images of retinal vasculature and choroidal vasculature with adjustable illumination intensity can comprise: providing a programmable illuminator including an optical assembly. The optical assembly can comprise: a backlight assembly comprising a VIS-NIR LED array, the VIS-NIR LED array comprising red LED channel, green LED channel, blue LED channel, and NIR LED channel configured to provide wavelength-selective illumination; diffuser plates or diffuser films configured to homogenize illumination emitted by the VIS-NIR LED array and provide spatially uniform illumination across an illumination aperture; and an LCD panel. The LCD panel can comprise: a first linear polarizer configured to receive the diffused illumination; a TFT layer adjacent to the first linear polarizer and configured to apply spatially resolved electric fields; a liquid crystal layer adjacent to the TFT layer and configured to modulate polarization state and transmissivity of incident light in response to control signals applied via the TFT layer; and a second linear polarizer positioned after the liquid crystal layer and configured to convert polarization modulation induced by the liquid crystal layer into intensity modulation of transmitted light. The LCD panel can be configured to selectively transmit the red LED channel, the green LED channel, and/or the blue LED channel from the VIS-NIR LED array to generate adjustable fixation targets comprising one or more point-illumination patterns. The method can further comprise: providing an image sensor comprising a camera; operably coupling the programmable illuminator with a controller via a DIO interface; operating the programmable illuminator in a Standby Mode, a Pre-Imaging/Alignment Mode, and an Imaging Mode, the Imaging Mode comprising a VIS Imaging Mode Type and an NIR Imaging Mode Type; selectively delivering illumination with the red LED channel, green LED channel, blue LED channel, and NIR LED channel through the LCD panel according to the selected Imaging Mode; and acquiring images of retinal vasculature and choroidal vasculature using the camera in coordination with the programmable illuminator. The Standby Mode can comprise a deactivated state of all VIS LED channels, NIR LED channel, and illuminator pixels, in which there is a complete absence of optical output from the optical assembly. The Pre-Imaging/Alignment Mode can comprise an activated state of the NIR LED channel and the red LED channel, the red LED channel being configured to pass through activated pixels of the LCD panel, reach a retina of a subject, and be visible to the subject, the NIR LED channel not being blocked by the LCD panel, such that the NIR illumination passes through the LCD panel and illuminate the entire retina. The VIS Imaging Mode Type can comprise: an activated state of the VIS LED channels with the NIR LED channel being deactivated; the activated VIS LED channels being pulsed; illumination from the pulsed VIS LED channels being delivered to a retina for a duration of about 200 milliseconds or less; and the camera, with an NIR filter, recording an image of retinal vasculature. An operator can control duration and power of the pulsed VIS LED channels via the controller to adjust brightness, contrast, and/or overall quality of the image. The NIR Imaging Mode Type can comprise: an activated state of only the NIR LED channel with the VIS LED channels being deactivated; the activated NIR LED channel being pulsed or continuous (i.e., not being pulsed); and the camera, without an NIR filter, recording an image of choroidal vasculature. The liquid crystal layer can be transparent to wavelengths above 750 nm (or about 750 nm), thereby permitting illumination from the NIR LED channel to pass without significant attenuation during the Pre-Imaging/Alignment Mode while the LCD panel generates visible-light fixation targets. The method can further comprise: applying localized optical stimulation to retinal tissue or choroidal tissue; coordinating the localized optical stimulation with image acquisition to assess functional characteristics of the retina; and using the assessment of neurovascular coupling to identify biomarkers associated with early-stage ocular disease. The camera (e.g., a wearable fundus camera) can also operate using either transpalpebral illumination or trans-pars plana illumination to expand the retinal region that can be imaged. The image sensor and the controller can be configured to perform high-speed spectral imaging, the high-speed spectral imaging can be configured to extract local hemodynamic information from retinal vasculature or choroidal vasculature, and the local hemodynamic information can comprise regional blood oxygenation as a function of time. The controller can be configured to store image data acquired across multiple imaging sessions, and the stored image data can be used to detect temporal changes in retinal vasculature or choroidal vasculature.

### BRIEF DESCRIPTION OF DRAWINGS

FIG. 1 shows an advanced functional fundus camera, according to an embodiment of the subject invention.

FIG. 2 shows a schematic view of a procedure for spectral imaging acquisition without stimulation, according to an embodiment of the subject invention.

FIG. 3 shows a schematic view of a procedure for spectral imaging acquisition with stimulation, according to an embodiment of the subject invention.

FIG. 4 shows a schematic view of a programmable illuminator, according to an embodiment of the subject invention.

FIG. 5 shows a Standby Mode, wherein all light-emitting diodes (LED) channels and illuminator pixels are deactivated, such that no illumination is delivered to the retina, according to an embodiment of the subject invention.

FIG. 6 shows a Pre-imaging/Alignment Mode, wherein both near-infrared (NIR) and red LED channels are activated, according to an embodiment of the subject invention.

FIG. 7 shows an Imaging Mode of a programmable illuminator, comprising two mode types: a visible (VIS) Imaging Mode Type and an NIR Imaging Mode Type, according to an embodiment of the subject invention.

### DETAILED DESCRIPTION

Embodiments of the subject invention provide novel and advantageous systems, devices, and methods for imaging vascular structures of retina and choroid, configured to acquire structural and functional information for identification and analysis of potential biomarkers associated with ocular and neurological diseases. The system provides simultaneous high-resolution imaging of both retinal and choroidal vascular layers, high-speed spectral imaging capable of extracting temporal data on regional blood oxygenation, and precise localized stimulation to study functional characteristics of the retina, thereby enabling detection of functional biomarkers for early disease evaluation. The system/device can be configured to acquire images from both eyes sequentially, and the system/device can be configured to acquire images from both eyes simultaneously (i.e., the images can be acquired either sequentially or simultaneously based on how the user uses the system, which is configured to perform either acquisition method). Also, the system/device can create a controlled dark environment for the patient, thereby facilitating examinations that traditionally require a dedicated darkroom (i.e., the system/device can mimic a dedicated darkroom).

An advanced functional fundus camera serves as a diagnostic imaging system configured to acquire high-resolution images of both vascular networks, enabling detailed visualization of ocular structures implicated in a wide range of ophthalmic diseases. The system is adapted to provide enhanced clarity of morphological features within retinal and choroidal vasculature, thereby facilitating detection of subtle vascular abnormalities associated with early stages of disease development. The system further comprises an illumination scheme configured to deliver both visible (VIS) and near-infrared (NIR) illumination and an adjustable fixation target to facilitate precise alignment of an eye of a subject and enhance gaze stability during imaging procedures.

Unlike conventional pupil-based fundus imaging systems, which typically provide limited fields of view that restrict visualization of peripheral retinal regions, the advanced functional fundus camera employs transpalpebral NIR spectral illumination, providing an enlarged retinal illumination area and enabling visualization of peripheral regions not readily captured using standard techniques. The wearable fundus camera can also operate using either transpalpebral illumination or trans-pars plana illumination to expand the retinal region that can be imaged. The system further incorporates high-speed spectral imaging functionality for localized assessment of hemodynamic parameters, including temporal variations in regional blood oxygenation, thereby supplementing structural imaging with physiologically relevant functional data. Additionally, the camera is configured to deliver localized stimulation to selected retinal regions to assess functional characteristics of the retina, enabling identification of vascular response characteristics that may serve as biomarkers for early disease detection.

The system is designed to enhance clinical usability by reducing operational complexity and minimizing user-dependent variability, supporting longitudinal imaging studies for early detection and monitoring of disease progression, while improving patient comfort, compliance, and examination efficiency.

Embodiments of the subject invention provide at least the following advantageous features: i) simultaneous multi-layer vascular imaging, wherein the system is configured to acquire high-resolution images of both retinal and choroidal vasculature simultaneously, enabling independent or coordinated analysis of structural features within each vascular network; ii) in the case where transpalpebral illumination or trans-pars plana illumination are used—expanded field of view, wherein trans-lid spectral illumination in the NIR wavelength region is configured to provide a larger retinal illumination area compared to conventional fundus imaging and optical coherence tomography, thereby enabling visualization of peripheral vascular regions and structural features that may not be captured using standard imaging techniques; iii) non-invasive functional assessment, wherein high-speed spectral imaging is adapted to measure hemodynamic parameters, including temporal changes in regional blood oxygenation, without the requirement for invasive contrast agents or exogenous dyes; iv) localized retinal functional assessment, wherein the device is configured to apply precise local stimulation to retinal and choroidal vasculature and measure vascular responses, thereby facilitating analysis of functional characteristics of the retina and enabling detection of potential functional biomarkers associated with neurological disorders; v) enhanced clinical workflow and accessibility, wherein the system is designed to reduce operational complexity, minimize user-dependent variability, and facilitate longitudinal imaging studies, thereby supporting routine clinical implementation and patient compliance; vi) integrated multimodal imaging, wherein structural and functional imaging modalities are combined within a single platform, reducing the need for multiple separate devices, streamlining examination procedures, and providing a unified dataset for comprehensive analysis; and vii) early detection capability, wherein the combination of high-resolution imaging, expanded field of view, and functional assessment is configured to detect subtle vascular and metabolic alterations that may precede clinical symptoms, thereby enabling earlier diagnostic evaluation of ocular, systemic, and neurological conditions.

The system is configured to design and construct a prototype programmable illuminator adapted as a critical optical component for the system, wherein the programmable illuminator is includes the following capabilities: 1) spectral flexibility, wherein the programmable illuminator is configured to provide independent control over illumination within VIS and NIR wavelength regions; 2) dynamic illumination modes, wherein the system supports pulsed illumination in red, green, blue, or white illumination with adjustable intensity, thereby enabling high-quality, non-mydriatic fundus imaging; and 3) programmable fixation target, wherein the programmable illuminator is adapted to generate an adjustable fixation target for precise alignment of the eye, facilitating acquisition of fundus images from selected retinal regions.

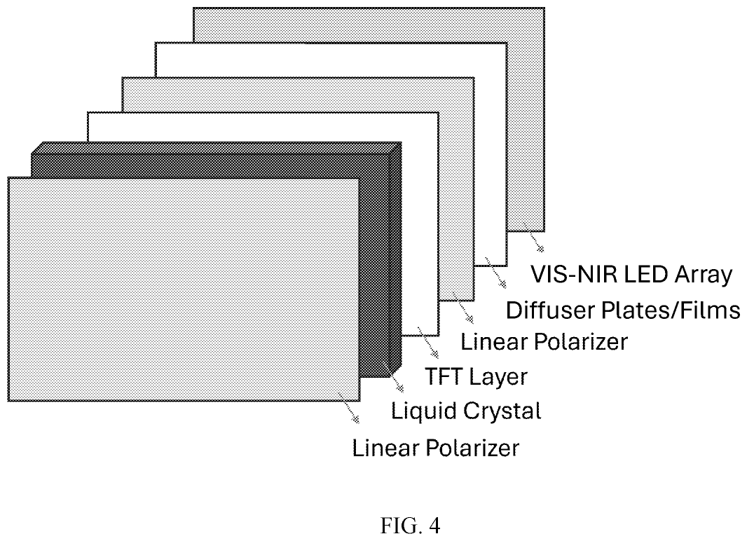

The design of the programmable illuminator is based on the structural principles of graphic liquid crystal display (LCD) monitors, as shown in FIG. 4. In place of a conventional white backlight, the programmable illuminator employs a backlight comprising red, green, blue, and NIR light-emitting diodes (LEDs), wherein individual control of the LEDs enables wavelength-selective illumination and efficient NIR illumination as required for the system. The two-dimensional (2D) illuminator operates by projecting point illumination onto the retina from each discrete point on its surface, thereby permitting selective illumination of specific retinal regions. The liquid crystal layer of the display remains substantially transparent to wavelengths above approximately 750 nanometers (nm), allowing NIR illumination to pass through unobstructed and enabling simultaneous use of NIR illumination for full retinal coverage during the pre-imaging phase while the LCD generates adjustable fixation targets using red, green, or blue illumination for precise eye alignment.

The operation of the programmable illuminator is implemented in three primary modes: (A) a Standby Mode, wherein the programmable illuminator is inactive or maintains minimal background illumination; (B) a Pre-Imaging/Alignment Mode, wherein NIR illumination is applied for full retinal coverage and the LCD generates adjustable fixation targets for eye alignment; and (C) an Imaging Mode, wherein the system delivers either visible-light or NIR illumination for acquisition of high-resolution fundus images.

In the Standby Mode, all LED channels and illuminator pixels are deactivated, such that no illumination is delivered to the retina.

In the Pre-Imaging/Alignment Mode, both NIR LED channel and red LED channel are activated, and a fixation target, such as a cross or other suitable pattern, is displayed on the liquid crystal layer of the programmable illuminator. Illumination from the red LED channel passes through the activated pixels and reaches the retina, allowing the fixation target to be perceived by the subject. Simultaneously, the LCD panel remains substantially transparent to NIR illumination, permitting NIR illumination to pass through the display and illuminate the entire retina. This configuration facilitates accurate eye alignment and focusing during the pre-imaging process, while minimizing visual discomfort and light sensitivity for the subject.

The Imaging Mode comprises two mode types: a VIS Imaging Mode Type, wherein all pixels of the liquid crystal layer are activated to allow maximum light transmission and pulsed visible illumination (red, green, blue, or white) is delivered to the retina for image acquisition. The programmable illuminator delivers pulsed visible illumination for a duration of approximately 200 milliseconds or less, and pulse duration and intensity are controllable by the controller through a software interface. The camera, in conjunction with an NIR filter, synchronously acquires retinal images to optimize brightness, contrast, and overall image quality; and an NIR Imaging Mode Type, wherein only the NIR LED channel is activated, providing continuous, non-pulsed illumination, and the camera acquires images of deeper ocular structures, such as the choroid, without the use of an NIR filter.

The programmable illuminator is configured to provide independent control of wavelength and intensity, and to display adjustable fixation targets, thereby enabling precise retinal illumination and alignment for functional and structural imaging. This illuminator is intended to enhance the versatility of the system while reducing system size and optical complexity, thereby facilitating advanced functional and structural assessments under controlled illumination conditions.

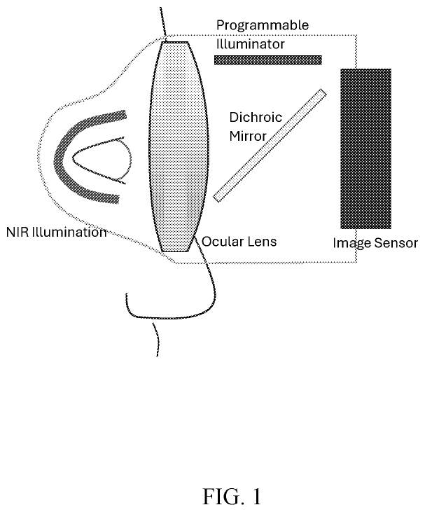

FIG. 1 shows an advanced functional fundus camera, according to an embodiment of the subject invention. Referring to FIG. 1, the advanced functional fundus camera can include an image sensor, an ocular lens disposed opposite from the image sensor. The advanced functional fundus camera can further include a programmable illuminator and/or a dichroic mirror. The dichroic mirror can be disposed between the ocular lens and the image sensor, and it can be disposed on a slant (see FIG. 1), such that the plane of the dichroic mirror is at an angle (e.g., in a range of from 20 degrees to 70 degrees, such as 45 degrees or about 45 degrees) with respect to a shortest imaginary line that connects the image sensor and the ocular lens. The programmable illuminator can be disposed between an upper portion of the ocular lens and an upper portion of the image sensor (see FIG. 1), and the plane of the programmable illuminator can be parallel to the shortest imaginary line connecting the image sensor and the ocular lens. The advanced functional fundus camera can include illumination on one side thereof (e.g., such that the ocular lens is between the illumination and the image sensor). The illumination can be, for example, NIR illumination. The dichroic mirror can reflect visible illumination but allow NIR illumination to transmit therethrough. The dichroic mirror can reflect both visible and NIR illumination at an efficiency of about 50%.

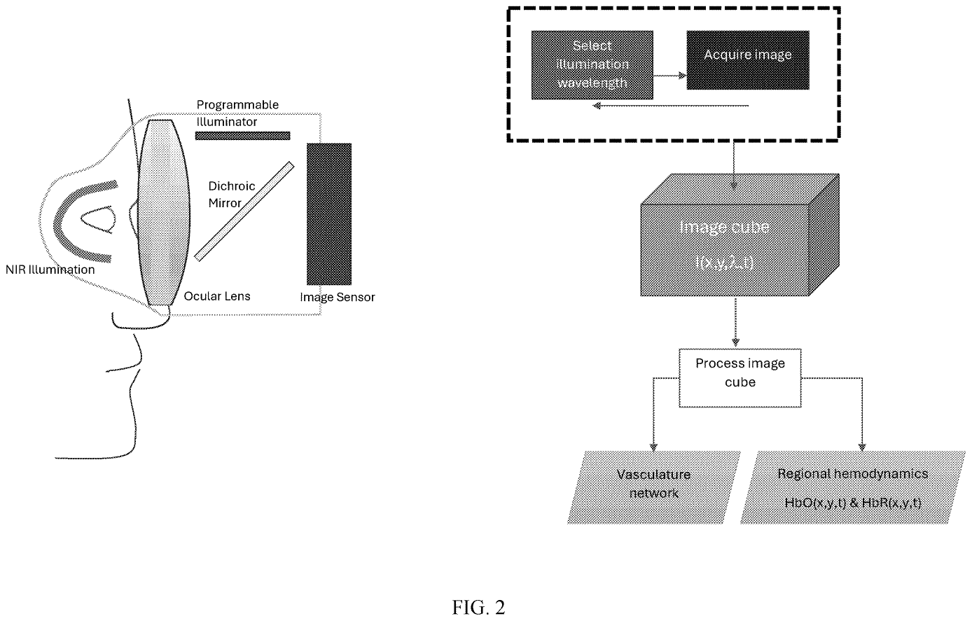

FIG. 2 shows a schematic view of a method for spectral imaging acquisition without stimulation, according to an embodiment of the subject invention. Referring to FIG. 2, the advanced functional fundus camera can be provided to the subject. A desired wavelength of illumination (e.g., NIR illumination) can be selected, the illumination can be provided, and an image can be acquired by the image sensor (e.g., camera). The image can be processed (e.g., using a processor) by a module (which may be stored on a (non-transitory) machine-readable medium in operable communication with the processor) that converts the image into an image cube and processes the image cube (e.g., I(x,y,λ,t), where x and y relate to position and t is time) to obtain a vasculature network of the eye of the subject and/or regional hemodynamics (e.g., HbO(x,y,t) and/or HbR(x,y,t), where HbO is oxygenated hemoglobin and HbR is deoxygenated hemoglobin) of the eye of the subject. The module can use artificial intelligence and/or machine learning. The processor and/or the machine-readable medium can be in operable communication with the advanced functional fundus camera (e.g., with the image sensor of the advanced functional fundus camera).

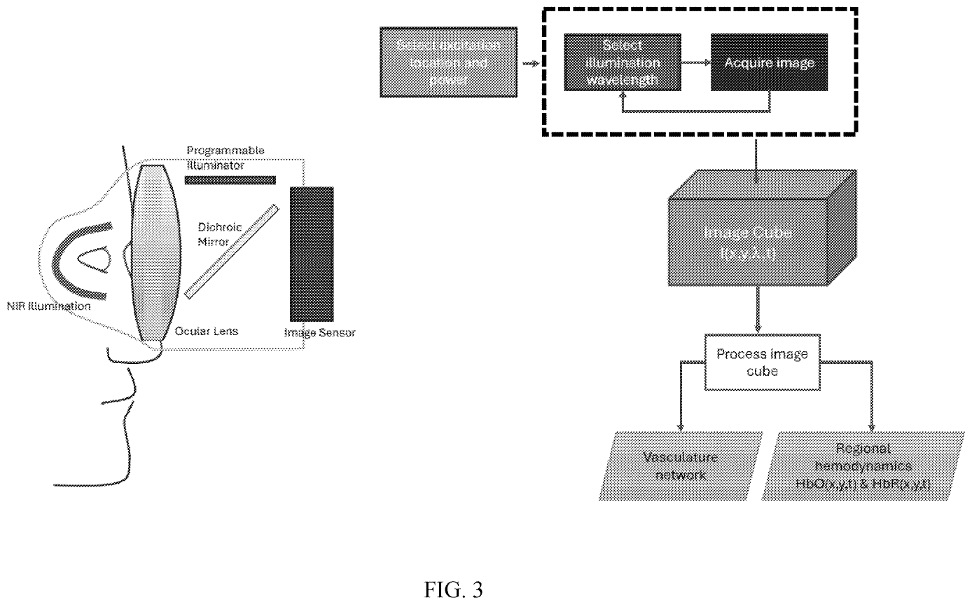

FIG. 3 shows a schematic view of a method for spectral imaging acquisition with stimulation, according to an embodiment of the subject invention. The method shown in FIG. 3 is similar to that shown in FIG. 2 except that stimulation/excitation is provided to the eye of the subject, so there can be an extra step of selecting the desired excitation location and/or power. The stimulation can be provided by the display. The elements/features/alternatives discussed in the previous paragraph apply for this method shown in FIG. 3 as well.

FIG. 4 shows a schematic view of the programmable illuminator, according to an embodiment of the subject invention. The programmable illuminator is architecturally based on structural and operational principles commonly employed in graphic LCD monitors, adapted for controlled ocular illumination applications.

The programmable illuminator includes a backlight assembly comprising a visible and near-infrared (VIS-NIR) LED array. The LED array includes red, green, blue, and NIR LED channels arranged to provide wavelength-selective illumination. Individual control of each LED type enables selective activation of visible wavelengths and efficient generation of NIR illumination as required for different operating modes of the system.

Positioned downstream of the LED array are diffuser plates or diffuser films configured to homogenize illumination emitted by the VIS-NIR LED array and provide spatially uniform illumination across an illumination aperture. The diffused illumination then passes through a first linear polarizer, which establishes a defined polarization state for subsequent modulation.

A thin-film transistor (TFT) layer is disposed after the first linear polarizer and is operable to apply spatially resolved electric fields to an adjacent liquid crystal layer. The liquid crystal layer is arranged to modulate the polarization state and transmissivity of the incident light in response to control signals applied via the TFT layer. By selectively driving regions of the liquid crystal layer, the programmable illuminator can dynamically control light transmission, including generation of fixation patterns, global transmissive states, or non-transmissive states.

A second linear polarizer is positioned after the liquid crystal layer and is oriented relative to the first linear polarizer to convert polarization modulation induced by the liquid crystal layer into intensity modulation of the transmitted light. Through coordinated control of the LED array and the liquid crystal layer, the programmable illuminator is capable of selectively transmitting, blocking, or patterning visible illumination while allowing NIR illumination to pass substantially unattenuated when desired.

The integrated configuration, comprising, in order, the VIS-NIR LED array, the diffuser plates or diffuser films, the first linear polarizer, the TFT layer, the liquid crystal layer, and the second linear polarizer, enables programmable, wavelength-selective, and spatially controllable illumination. This design supports multiple operational modes, including the Standby Mode, the Pre-Imaging/Alignment Mode, and the Imaging Mode, while maintaining a compact and integrable form factor suitable for ophthalmic imaging systems.

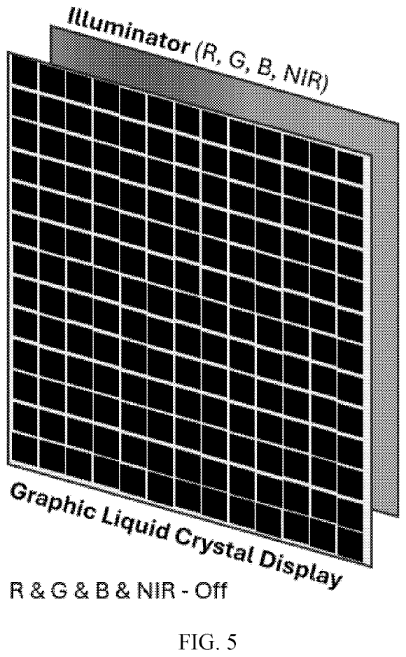

FIG. 5 shows the Standby Mode of the programmable illuminator in which illumination output is disabled. As shown, visible-light and NIR light sources are inactive, and the liquid crystal layer is in a non-transmissive condition such that light is not directed toward the eye of the subject. In this state, no illumination is provided to the retinal region, representing an inactive or idle configuration of the programmable illuminator.

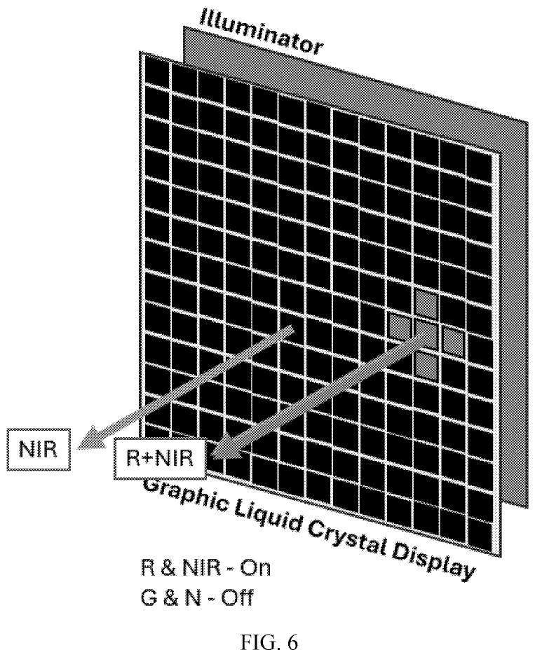

FIG. 6 shows the Pre-imaging/Alignment Mode in which both a NIR illumination channel and a visible-light illumination channel are active. In this mode, a fixation pattern is generated on the liquid crystal display positioned in the illumination path. Visible light from the red illumination source is selectively transmitted through activated regions of the display to form the fixation pattern visible to the eye of the subject. At the same time, the display permits NIR light to pass unattenuated, allowing the NIR illumination to reach the retinal surface. The combined illumination enables visual fixation by the subject while providing uniform NIR illumination for alignment and focusing.

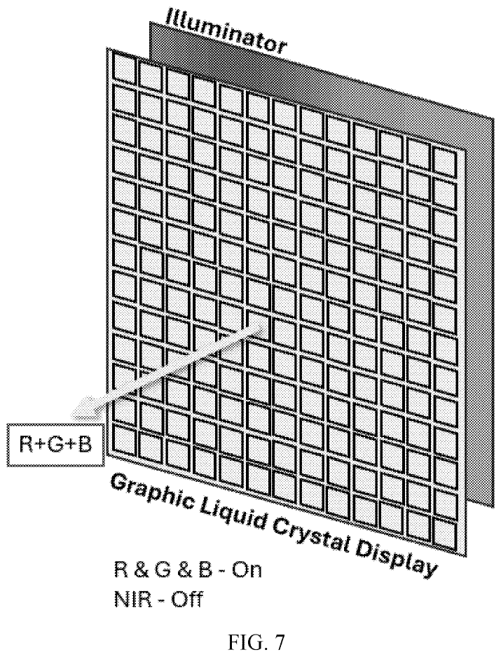

FIG. 7 shows the Imaging Mode of a programmable illuminator that includes the VIS Imaging Mode Type and the NIR Imaging Mode Type. In the VIS Imaging Mode Type, the liquid crystal layer is shown in a fully transmissive state while visible illumination is applied in a pulsed manner for retinal image capture, with image acquisition coordinated by the camera. In the NIR Imaging Mode Type, the figure depicts activation of NIR light sources to provide illumination for imaging underlying ocular structures, with the camera configured to receive NIR light. Together, the illustrated configurations represent alternative imaging arrangements supported by the programmable illuminator.

With respect to superior field of view, the trans-lid spectral illumination in the NIR wavelength region delivers a significantly larger area of illumination compared to related art techniques like optical coherence tomography (OCT) and standard fundus photography. This expanded field of view captures peripheral vascular changes that may be missed by more narrowly focused imaging methods.

With respect to non-invasive functional assessment, while technologies like indocyanine green angiography require invasive dye injection, embodiments of the subject invention offer non-invasive spectral imaging to extract hemodynamic information, particularly time dependent regional blood oxygenation. This provides functional data without patient discomfort or potential adverse reactions to contrast agents.

With respect to novel neurovascular coupling assessment, unlike related art technologies, embodiments of the subject invention have the ability to apply local stimulation to study neurovascular coupling in both retinal and choroidal vasculature, representing a significant advancement. This functionality has the potential to identify entirely new biomarkers for neurological disease diagnosis that related art technologies cannot detect.

With respect to enhanced clinical utility and accessibility, compared to complex systems like OCT and dynamic visual acuity (DVA) that require specialized training, embodiments of the subject invention offer exceptional ease of use, making them accessible for widespread clinical implementation. This accessibility facilitates longitudinal studies that are essential for early disease detection and monitoring progression across a spectrum of neurological disorders.

With respect to multimodal capability in a single platform, while related art technologies require multiple imaging modalities (OCT for structure, DVA for function, etc.), embodiments of the subject invention integrate structural and functional assessment in a single platform, streamlining workflow, reducing examination time, and providing a more comprehensive dataset for analysis.

With respect to superior early detection potential, the combination of high-resolution imaging expanded field of view, and functional assessment capabilities positions embodiments of the subject invention to detect subtle vascular changes that may precede clinical symptoms of neurological disorders, potentially offering earlier diagnostic capabilities than related art technologies.

Embodiments of the subject invention provide cutting-edge diagnostic imaging systems specifically designed/configured to capture high-resolution images of both retinal and choroidal vasculature. This represents a significant advancement in ophthalmic imaging technology, offering comprehensive visualization of ocular structures that play critical roles in numerous eye diseases.

Embodiments of the subject invention provide exceptional clarity in visualizing the morphological characteristics of both retinal and choroidal vascular networks. This detailed imaging capability enables clinicians to identify subtle vascular abnormalities that may indicate early disease development.

Unlike related art fundus cameras, embodiments of the subject invention can utilize transpalpebral spectral illumination in the NIR wavelength region. This approach delivers a significantly larger area of illumination to the retina, resulting in an expanded field of view of fundus imaging. The broader perspective allows for a more comprehensive assessment of peripheral retinal regions that are often missed with standard imaging techniques.

Embodiments of the subject invention also provide the high-speed spectral imaging capability to extract crucial hemodynamic information, particularly regional blood oxygenation data as a function of time. This functional assessment capability adds a valuable dimension to the structural imaging, providing insights into tissue perfusion and metabolic activity.

Embodiments of the subject invention can also be configured to apply precise local stimulation to study neurovascular coupling in both retinal and choroidal vasculature. This functionality enables the identification of potential new biomarkers for early disease detection and diagnosis, opening avenues for earlier therapeutic intervention.

Designed with clinical workflow in mind, embodiments of the subject invention offer remarkable ease of use, reducing the technical expertise required for operation. This accessibility facilitates longitudinal studies that are essential for early disease detection and monitoring disease progression over time. The simplified operation also increases patient compliance and reduces examination time.

Despite their considerable value, related art retinal imaging technologies exhibit significant limitations. While OCT provides high-resolution structural images, optical coherence tomography angiography (OCTA) visualizes vasculature networks, and other modalities measure blood flow or hemodynamics individually, no related art technology offers simultaneous vascular and hemodynamic imaging with the capability to monitor regional hemodynamic changes via local light stimulation. Additionally, these related art systems are typically expensive and require specialized training to operate, substantially limiting their accessibility in clinical settings and creating barriers to the longitudinal studies essential for early disease detection and progression monitoring. These limitations highlight the critical need for more integrated, user-friendly imaging solutions that combine multiple assessment capabilities in a single, accessible platform.

To address these technological gaps, embodiments of the subject invention provide diagnostic imaging systems, devices, and methods that can simultaneously capture high-resolution images of both retinal and choroidal vasculature. Transpalpebral NIR spectral illumination can be employed to significantly expand the field of view, revealing peripheral retinal regions typically missed by conventional imaging. The high-speed spectral imaging capabilities can extract crucial hemodynamic information including regional blood oxygenation over time, while the unique ability to apply precise local stimulation allows for neurovascular coupling assessment, potentially revealing new biomarkers for early disease detection. Designed with clinical accessibility in mind, embodiments of the subject invention can dramatically reduce the expertise required for operation, facilitating the longitudinal studies essential for early diagnosis and disease monitoring while improving patient compliance through simplified, efficient examination protocols.

Embodiments of the subject invention provide significant advantages to the field of ocular and neurological diagnostics. First, the unique combination of simultaneous vascular and hemodynamic imaging with neurovascular coupling assessment capabilities enables researchers to uncover novel biomarkers for both retinal and neurological diseases, potentially revolutionizing early diagnosis protocols. Second, the exceptional ease of use and simplified operation dramatically reduce barriers to implementing comprehensive longitudinal studies, facilitating consistent tracking of disease occurrence and progression while increasing patient compliance through shorter examination times. Third, by generating standardized, multimodal imaging data across diverse populations, the foundation for artificial intelligence (AI)-enabled medicine is provided, powering machine learning algorithms capable of detecting subtle disease patterns, predicting progression, and personalizing treatment approaches. This ultimately positions embodiments of the subject invention at the critical intersection of ophthalmology, neurology, and precision medicine.

The advanced functional fundus camera explores new biomarkers for eye and brain diseases. It features simultaneous high-resolution imaging of both retinal and choroidal vasculature, transpalpebral NIR spectral illumination that significantly expands the field of view to capture peripheral retinal regions, and high-speed spectral imaging that extracts critical temporal data on regional blood oxygenation. Additionally, the device facilitates precise local stimulation to study neurovascular coupling, potentially revealing new biomarkers for early disease detection.

Development of the advanced functional fundus camera integrated advanced optical engineering with clinical workflow considerations, focusing on the bilateral system design, spectral illumination implementation, hemodynamic data analysis algorithms, and optimization for clinical accessibility. This resulted in enhanced diagnostic capabilities through visualization of previously unobservable vascular changes, identification of novel retinal biomarkers for neurodegenerative conditions, improved clinical workflow through simplified operation, and advancement of research investigating relationships between retinal changes and systemic or neurological conditions. Embodiments of the subject invention represent a significant advancement in ophthalmic imaging with far-reaching implications for both clinical practice and medical research.

Embodiments of the subject invention provide a focused technical solution to the focused technical problem of how to capture non-mydriatic fundus images of retinal vasculature and choroidal vasculature with adjustable illumination intensity. The solution is provided by the programmable illuminator including the optical assembly comprising the backlight assembly comprising the VIS-NIR LED array, the VIS-NIR LED array comprising red LED channel, green LED channel, blue LED channel, and NIR LED channel configured to provide wavelength-selective illumination, and the LCD panel configured to selectively transmit illumination from the LEDs to generate adjustable fixation targets comprising one or more point-illumination patterns. Embodiments can operate the programmable illuminator in the Standby Mode, the Pre-Imaging/Alignment Mode, and the Imaging Mode, the Imaging Mode comprising the VIS Imaging Mode Type and the NIR Imaging Mode Type, and selectively delivering illumination through the LCD panel according to the selected Imaging Mode. Images of retinal vasculature and choroidal vasculature are acquired using the camera in coordination with the programmable illuminator. Embodiments further provide the practical application of enabling (and/or performing and/or controlling) structural and functional assessment of ocular vasculature, including imaging peripheral retinal regions, extracting local hemodynamic data such as regional blood oxygenation over time, coordinating localized optical stimulation with image acquisition to assess neurovascular coupling, and/or storing image data across multiple sessions to detect temporal changes indicative of early-stage ocular disease. Embodiments can improve specific technologies, including retinal imaging devices and ophthalmic diagnostic instruments, by enabling (and/or performing and/or controlling) more precise alignment, higher-resolution imaging, functional vascular assessment, and enhanced early detection of ocular pathologies.

The methods and processes described herein can be embodied as code and/or data. The software code and data described herein can be stored on one or more machine-readable media (e.g., computer-readable media), which may include any device or medium that can store code and/or data for use by a computer system. When a computer system and/or processor reads and executes the code and/or data stored on a computer-readable medium, the computer system and/or processor performs the methods and processes embodied as data structures and code stored within the computer-readable storage medium.

It should be appreciated by those skilled in the art that computer-readable media include removable and non-removable structures/devices that can be used for storage of information, such as computer-readable instructions, data structures, program modules, and other data used by a computing system/environment. A computer-readable medium includes, but is not limited to, volatile memory such as random access memories (RAM, DRAM, SRAM); and non-volatile memory such as flash memory, various read-only-memories (ROM, PROM, EPROM, EEPROM), magnetic and ferromagnetic/ferroelectric memories (MRAM, FeRAM), and magnetic and optical storage devices (hard drives, magnetic tape, CDs, DVDs); network devices; or other media now known or later developed that are capable of storing computer-readable information/data. Computer-readable media should not be construed or interpreted to include any propagating signals. A computer-readable medium of embodiments of the subject invention can be, for example, a compact disc (CD), digital video disc (DVD), flash memory device, volatile memory, or a hard disk drive (HDD), such as an external HDD or the HDD of a computing device, though embodiments are not limited thereto. A computing device can be, for example, a laptop computer, desktop computer, server, cell phone, or tablet, though embodiments are not limited thereto.

When the term module is used herein, it can refer to software and/or one or more algorithms to perform the function of the module; alternatively, the term module can refer to a physical device configured to perform the function of the module (e.g., by having software and/or one or more algorithms stored thereon).

When ranges are used herein, combinations and subcombinations of ranges (including any value or subrange contained therein) are intended to be explicitly included. When the term “about” or “approximately” is used herein, in conjunction with a numerical value, it is understood that the value can be in a range of 95% of the value to 105% of the value, i.e. the value can be +/−5% of the stated value. For example, “about 1 kg” means from 0.95 kg to 1.05 kg.

It should be understood that the examples and embodiments described herein are for illustrative purposes only and that various modifications or changes in light thereof will be suggested to persons skilled in the art and are to be included within the spirit and purview of this application.

All patents, patent applications, provisional applications, and publications referred to or cited herein are incorporated by reference in their entirety, including all figures and tables, to the extent they are not inconsistent with the explicit teachings of this specification.

What is claimed is:

1. A system for capturing non-mydriatic fundus images of retinal vasculature and choroidal vasculature with adjustable illumination intensity, the system comprising:
a programmable illuminator;
an image sensor configured to capture fundus images; and
a controller operably coupled to the programmable illuminator and the image sensor, the controller comprising one or more processors and a non-transitory machine-readable medium storing instructions that, when executed, cause the system to:
operate in a pre-imaging alignment mode in which an near-infrared (NIR) wavelength channel provides alignment illumination;
operate in an imaging mode including:
(i) a visible imaging mode in which at least one visible wavelength channel provides imaging illumination, and
(ii) an NIR imaging mode in which the NIR wavelength channel provides imaging illumination; and
synchronize activation of a multi-channel light source with exposure timing of the image sensor,
the controller being configured to control illumination intensity of the multi-channel light source at levels sufficient to permit fundus imaging without dilating pupils of a subject, and
a spatial light modulator selectively modulating illumination from the multi-channel light source to generate fixation targets while the multi-channel light source provides imaging illumination.

2. The system according to claim 1, further comprising the multi-channel light source,
the multi-channel light source comprising a plurality of light-emitting diode (LED) channels having the at least one visible wavelength channel and the at least one NIR wavelength channel.

3. The system according to claim 2, further comprising one or more optical homogenizing elements positioned to receive illumination from the multi-channel light source and to provide spatially uniform illumination.

4. The system according to claim 3, further comprising the spatial light modulator,
the spatial light modulator being positioned to receive illumination from the multi-channel light source and configured to spatially and intensity-modulate the illumination to generate one or more fixation targets.

5. The system according to claim 1, the system further comprising a stimulation subsystem configured to apply localized optical stimulation to retinal tissue or choroidal tissue, the controller being configured to coordinate the localized optical stimulation with image acquisition to assess functional characteristics of a retina of a subject, the assessment enabling identification of biomarkers associated with early-stage ocular disease.

6. The system according to claim 1, further comprising an NIR LED illuminator configured to deliver transpalpebral illumination to an eye of the subject, the transpalpebral illumination from the NIR LED illuminator providing an expanded retinal illumination area relative to pupil-based fundus illumination, the expanded illumination area enabling imaging of peripheral retinal regions.

7. The system according to claim 1, the image sensor and the controller being configured to perform high-speed spectral imaging, the high-speed spectral imaging being configured to extract local hemodynamic information from retinal vasculature or choroidal vasculature, the local hemodynamic information comprising regional blood oxygenation as a function of time.

8. The system according to claim 1, the controller being configured to store image data acquired across multiple imaging sessions, the stored image data being used to detect temporal changes in retinal vasculature or choroidal vasculature.

9. The system according to claim 1, the system being configured to obtain images from both eyes of the subject sequentially, and
the system being configured to obtain images from both eyes of the subject simultaneously.

10. The system according to claim 1, the system being configured to provide a controlled dark environment that mimics a dedicated darkroom.

11. A method for capturing non-mydriatic fundus images of retinal vasculature and choroidal vasculature with adjustable illumination intensity, the method comprising:
providing a system comprising a programmable illuminator, an image sensor configured to capture fundus images, and a controller operably coupled to the programmable illuminator and the image sensor, the controller comprising one or more processors and a non-transitory machine-readable medium storing instructions;
executing the instructions to cause the system to:
operate in a pre-imaging alignment mode in which an near-infrared (NIR) wavelength channel provides alignment illumination;
operate in an imaging mode including:
(i) a visible imaging mode in which at least one visible wavelength channel provides imaging illumination, and
(ii) an NIR imaging mode in which the NIR wavelength channel provides imaging illumination; and
synchronize activation of a multi-channel light source with exposure timing of the image sensor; and
using a spatial light modulator to selectively modulate illumination from the multi-channel light source to generate fixation targets while the multi-channel light source provides imaging illumination,
the controller being configured to control illumination intensity of the multi-channel light source at levels sufficient to permit fundus imaging without dilating pupils of a subject.

12. The method according to claim 11, the multi-channel light source comprising a plurality of light-emitting diode (LED) channels having the at least one visible wavelength channel and the at least one NIR wavelength channel.

13. The method according to claim 12, the system further comprising one or more optical homogenizing elements positioned to receive illumination from the multi-channel light source and to provide spatially uniform illumination,
the spatial light modulator being positioned to receive illumination from the multi-channel light source and configured to spatially and intensity-modulate the illumination to generate one or more fixation targets.

14. The method according to claim 11, further comprising:
applying localized optical stimulation to retinal tissue or choroidal tissue;
coordinating the localized optical stimulation with image acquisition to assess neurovascular coupling; and
using the assessment of functional characteristics of a retina of the subject to identify biomarkers associated with early-stage ocular disease.

15. The method according to claim 11, further comprising using an NIR LED illuminator to deliver transpalpebral illumination to an eye of a subject, the transpalpebral illumination from the NIR LED illuminator providing an expanded retinal illumination area relative to pupil-based fundus illumination, the expanded illumination area enabling imaging of peripheral retinal regions.

16. The method according to claim 11, the image sensor and the controller being configured to perform high-speed spectral imaging, the high-speed spectral imaging being configured to extract local hemodynamic information from retinal vasculature or choroidal vasculature, the local hemodynamic information comprising regional blood oxygenation as a function of time.

17. The method according to claim 11, the controller being configured to store image data acquired across multiple imaging sessions, the stored image data being used to detect temporal changes in retinal vasculature or choroidal vasculature.

18. The method according to claim 11, further comprising obtaining images from both eyes of the subject sequentially or simultaneously.

19. The method according to claim 11, further comprising using the system to provide a controlled dark environment that mimics a dedicated darkroom.

20. A system for capturing non-mydriatic fundus images of retinal vasculature and choroidal vasculature with adjustable illumination intensity, the system comprising:
a programmable illuminator;
an image sensor configured to capture fundus images;
a multi-channel light source;
one or more optical homogenizing elements positioned to receive illumination from the multi-channel light source and to provide spatially uniform illumination;
a spatial light modulator positioned to receive illumination from the multi-channel light source and configured to spatially and intensity-modulate the illumination to generate one or more fixation targets; and
a controller operably coupled to the programmable illuminator and the image sensor, the controller comprising one or more processors and a non-transitory machine-readable medium storing instructions that, when executed, cause the system to:
operate in a pre-imaging alignment mode in which an near-infrared (NIR) wavelength channel provides alignment illumination;
operate in an imaging mode including:
(i) a visible imaging mode in which at least one visible wavelength channel provides imaging illumination, and
(ii) an NIR imaging mode in which the NIR wavelength channel provides imaging illumination; and
synchronize activation of the multi-channel light source with exposure timing of the image sensor,
the controller being configured to control illumination intensity of the multi-channel light source at levels sufficient to permit fundus imaging without dilating pupils of a subject, and
the spatial light modulator selectively modulating illumination from the multi-channel light source to generate fixation targets while the multi-channel light source provides imaging illumination,
the multi-channel light source comprising a plurality of light-emitting diode (LED) channels having the at least one visible wavelength channel and the at least one NIR wavelength channel,
the system further comprising a stimulation subsystem configured to apply localized optical stimulation to retinal tissue or choroidal tissue, the controller being configured to coordinate the localized optical stimulation with image acquisition to assess functional characteristics of a retina of a subject, the assessment enabling identification of biomarkers associated with early-stage ocular disease,
the system further comprising an NIR LED illuminator configured to deliver transpalpebral illumination to an eye of the subject, the transpalpebral illumination from the NIR LED illuminator providing an expanded retinal illumination area relative to pupil-based fundus illumination, the expanded illumination area enabling imaging of peripheral retinal regions,
the image sensor and the controller being configured to perform high-speed spectral imaging, the high-speed spectral imaging being configured to extract local hemodynamic information from retinal vasculature or choroidal vasculature, the local hemodynamic information comprising regional blood oxygenation as a function of time,
the controller being configured to store image data acquired across multiple imaging sessions, the stored image data being used to detect temporal changes in retinal vasculature or choroidal vasculature,
the system being configured to obtain images from both eyes of the subject sequentially,
the system being configured to obtain images from both eyes of the subject simultaneously, and
the system being configured to provide a controlled dark environment that mimics a dedicated darkroom.
